(19) United States
(12) Patent Application Publication
(10) Pub. No.: US 2005/0085788 A1
(43) Pub. Date: Apr. 21, 2005

(54) Apparatus for patients of erectile dysfunction

(76) Inventors: Kazuyuki Tomosada, Tokyo (JP); Yuiro Hata, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/921,881

(22) Filed: Aug. 20, 2004

(30) Foreign Application Priority Data
Aug. 20, 2003 (JP) 2003-289282
Aug. 20, 2003 (JP) 2003-289283

(51) Int. Cl. A61M 31/00

(52) U.S. Cl. 604/500

(57) ABSTRACT

The invention provides an external device for treatment of erectile dysfunction or impotence that enables an impotent to get through with normal sexual intercourse. The external device is molded integrally of a silicone rubber having a Shore A hardness of 5±2 as measured according to ASTM-D412.

### BACKGROUND OF THE INVENTION

The present invention relates generally to apparatus for patients of erectile dysfunction, and more specifically to an external device that enables those who suffer from erectile dysfunction or impotence to sustain an erection for normal sexual intercourse.

It has recently been estimated that there are increasing numbers of impotents. So far, such patients have been treated with silicone implantation. However, silicone causes some complications upon exposed to view, and is still insufficient for treatment of erectile dysfunction or impotence.

### SUMMARY OF THE INVENTION

In view of such prior art problems as described above, an object of the invention is to provide an external device that, upon attachment, can keep the penis virtually erected long enough to get through with normal sexual intercourse.

According to the invention, this object is achieved by the provision of an external device for treatment of erectile dysfunction or impotence, which is integrally molded of a silicone rubber, wherein said silicone rubber has a Shore A hardness of 5±2 as measured according to ASTM-D412.

Preferably, the external device of the invention should further comprise a head member, wherein:

said head member has a root and a head integrally molded of said silicone rubber, each in a cylindrical form,

said head has a streamlined spindle-like contour with a maximum diameter larger than said root, and is provided in its tip with an opening through which emitted semen is to be discharged, and

said head has a first hollow portion to be fitted over the penis glans and a second hollow portion in communication with said first hollow portion and said opening.

Preferably, the head member should have a full length of 80 to 300 mm, said head should have a maximum diameter of 30 to 45 mm, and said second hollow portion should have a length of 8 to 20 mm and a diameter of 8 to 15 mm, with a distance of 8 to 20 mm from said second hollow portion to a solid portion of said head.

Preferably, the external device of the invention should further comprise a shaft member molded integrally of said silicone rubber.

Preferably, said shaft member should further comprise protuberances spotted over the outer and inner surfaces.

### BRIEF DESCRIPTION OF THE DRAWINGS

FIG. 1 is a front view of the first embodiment of the head member.

FIG. 2 is a sectional view of the first embodiment of the head member.

FIG. 3 is a front view of the second embodiment of the head member.

FIG. 4 is a sectional view of the second embodiment of the head member.

FIG. 5 is a front view of the third embodiment of the head member.

FIG. 6 is a sectional view of the third embodiment of the head member.

FIGS. 7 and 8 are a front and an end view of one embodiment of the shaft member, respectively.

### EXPLANATION OF THE PREFERRED EMBODIMENTS

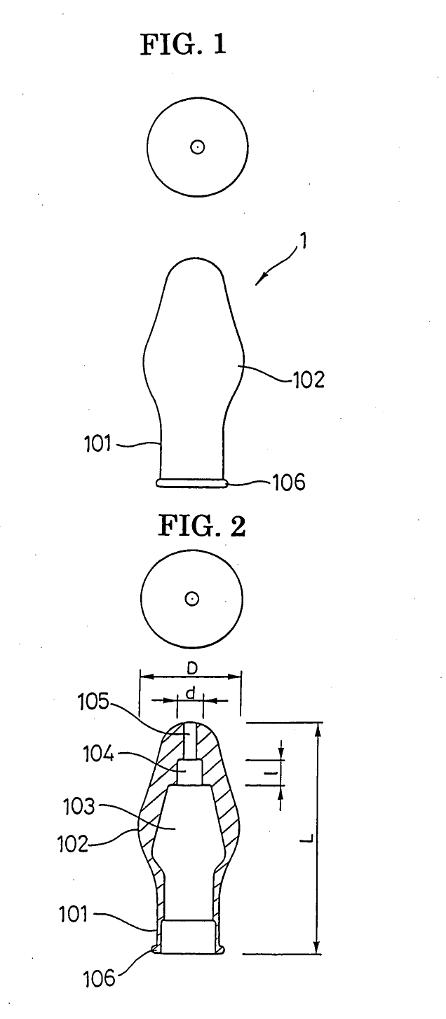

FIGS. 1 and 2 are a front view and a sectional view of the first embodiment of the invention, respectively. As depicted in FIGS. 1 and 2, the external device of the invention comprises a cylindrical form of head member generally molded integrally of a silicone rubber. The head member has a root 101 and a head 102, each in a cylindrical form. The root 101 has a length and diameter determined in such a way as to be well fitted over the penis that suffers from erectile dysfunction or impotence.

The head 102 is similar in shape to the penis glans, and has a length and diameter determined in such a way to effect smooth insertion and piston movement. In other words, the head 102 has a streamlined spindle-like contour. The tip of the head 102 is provided with an opening 105 for discharge of emitted semen. The external device is provided on the root side with two hollow portions 103 and 104, of which the first hollow portion 103 has an outer diameter and shape enough to be well fitted over the penis glans. The second hollow portion 104 is vitally important for keeping the strength of the head 102 and gaining weight reductions. Specifically, the second hollow portion 104 has a length l of the order of 8 to 20 mm and a diameter d of the order of 8 to 15 mm.

Then, the external device of the invention has a full length L of the order of 80 to 300 mm; the root has a thickness of the order of 2 to 3 mm; and the head 102 has a maximum diameter D of the order of 30 to 45 mm. Preferably, the length from the second hollow portion 104 to a solid portion is of the order of 8 to 20 mm. This ensures sufficient strength and weight reductions.

Such an integral external device is formed of a silicone rubber that should preferably have a Shore A hardness of 5±2, as measured pursuant to ASTM-D412. Medical silicones could be used for that silicone. The silicone rubber used could comprise 80 to 95% of a silicone polymer, 5 to 20% of silica and 0.05 to 1% of titania with the rest comprising suitable amounts of phthalocyanine dyes, carbon blacks, etc.

It is noted that in the embodiment shown, the lowermost end of the root is provided with a collar 106 for winding-up purposes.

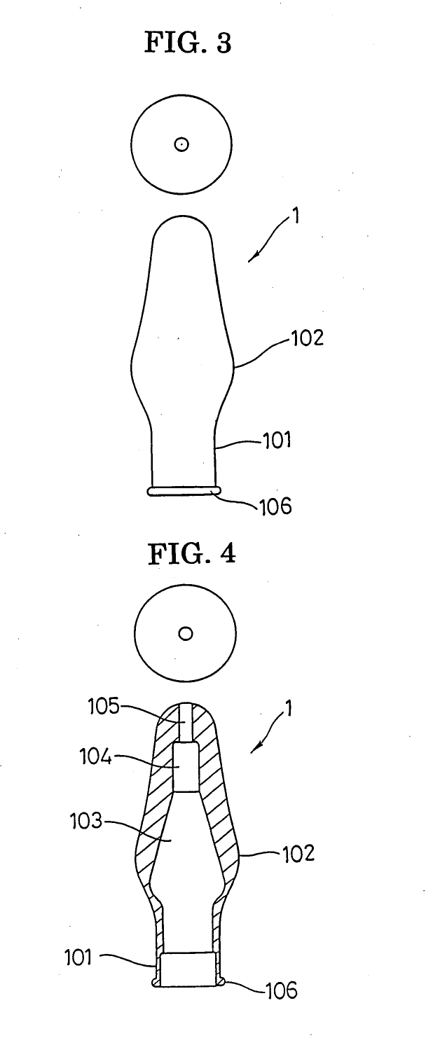

The second embodiment of the invention shown in FIGS. 3 and 4 is substantially the same as the first embodiment of the invention, and so is explained only by reference to reference numbers.

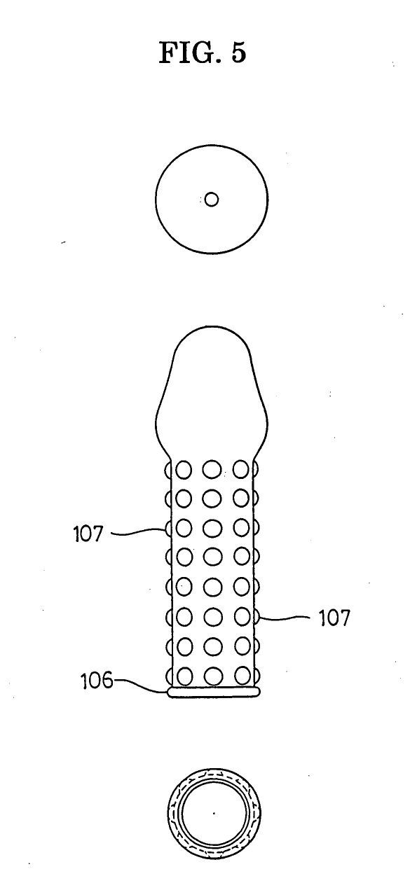

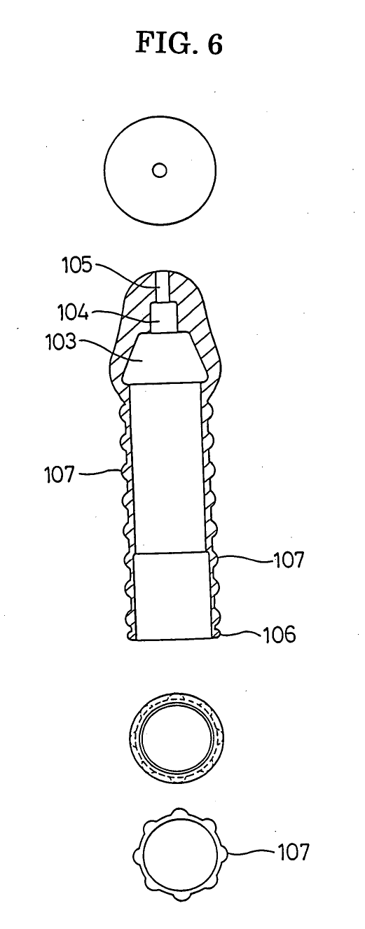

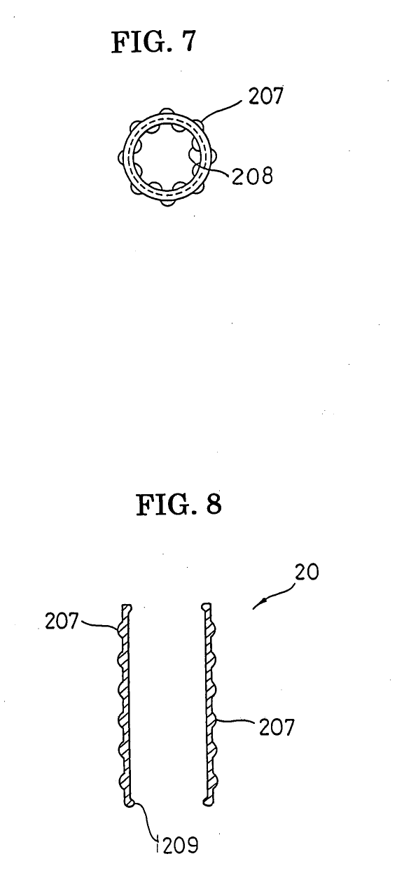

In the third embodiment of the invention depicted in FIGS. 5 and 6, a plurality of protuberances 107 of 1 to 2 mm in height are spotted around the root of the external device as explained above. Each protuberance 107 works to enhance sexual excitement during sexual intercourse.

For use, such a shaft member 20 as shown in FIG. 8 is first provided. This shaft member 20 again formed of silicone is configured in a cylindrical form having a given length and outside diameter as well as the same thickness as explained above. Specifically, the shaft member 20 has an outside diameter of 25 to 35 mm and an inside diameter of 20 to 28 mm, and on the outer and inner surfaces there are provided a plurality of protuberances 207 and 208. Although the protuberances 207 on the outer surface work as mentioned above, it is understood that the protuberances 208 on the inner surface work to enhance contact with the penis. On the inside of the rearmost end of the shaft member there is provided a ring-like rib 209 to enhance contact with the penis, and the outside thereof is somewhat R-configured in such a way as to be better fitted to the head member.

In use, the shaft member is rolled up to the end side. The rolled-up shaft member is opened by the first and second fingers of both hands for attachment to the root of the penis glans. Thereafter, the shaft member is rolled down along the penis. Then, the head member is attached in place. In this case, the head member is fully rolled up from the root end to the head end, whereupon it is fitted over the end of the penis glans, and then rolled down toward the shaft member. At the junction of the shaft member and the head member, the shaft member is positioned over the head member.

With the inventive external device attached in place, the penis can be kept virtually erected to get through with normal sexual intercourse.

### EXAMPLE

Such an external device as depicted in FIGS. 1 and 2 was fabricated of the silicone rubber to demonstrate the advantages of the invention. The external device was attached to the penis of one inventor of this invention.

More specifically, one inventor of the invention, Kazuyuki TOMOSADA was made to wear the external device on for diagnosis by another inventor, Dr. Yuiro HATA.

The patient diagnosed as impotency was once subjected to silicone implantation. Still, impotency was not ameliorated at all due to exposure of the implant. However, the attachment of the novel external device of the invention made a great contribution to amelioration; the patient could get through with normal sexual intercourse at any desired position, including of emission.

1. An external device for treatment of erectile dysfunction, which is molded integrally of a silicone rubber, wherein said silicone rubber has a Shore A hardness of 5±2 as measured according to ASTM-D412.

2. The external device according to claim 1, which further comprises a head member, wherein:
said head member has a root and a head molded integrally of said silicone rubber, each in a cylindrical form,
said head has a streamlined spindle-like contour with a maximum diameter larger than said root, and is provided in a tip with an opening through which emitted semen is to be discharged, and
said head has a first hollow portion to be fitted over the penis glans and a second hollow portion in communication with said first hollow portion and said opening.

3. The external device according to claim 1, wherein said head member has a full length of 80 to 300 mm, said head has a maximum diameter of 30 to 45 mm, and said second hollow portion has a length of 8 to 20 mm and a diameter of 8 to 15 mm, with a distance of 8 to 20 mm from said second hollow portion to a solid portion of said head.

4. The external device according to any one of claims 1 to 3, which further comprise a shaft member molded integrally of said silicone rubber.

5. The external device according to claim 4, wherein said shaft member further comprises protuberances spotted over outer and inner surfaces thereof.
